(19) United States
(12) Patent Application Publication
(10) Pub. No.: US 2018/0298314 A1
(43) Pub. Date: Oct. 18, 2018

(54) Photobioreactor, System and Method of Use

(71) Applicant: Algenol Biofuels Switzerland GmbH, Zug (CH)

(72) Inventors: Oliver Ashley, Fort Myers, FL (US); Omatoyo Dalrymple, Kansas City, MO (US); Harlan L. Miller, Fort Myers, FL (US); Lindsey Smith, Denver, CO (US); Thomas Stone, Fort Myers, FL (US)

(73) Assignee: Algenol Biofuels Switzerland GmbH, Zug (CH)

(21) Appl. No.: 16/010,613

(22) Filed: Jun. 18, 2018

Related U.S. Application Data
(63) Continuation of application No. PCT/US2016/068153, filed on Dec. 21, 2016.
(60) Provisional application No. 62/270,458, filed on Dec. 21, 2015.

(51) Int. Cl. C12M 1/00; C12M 1/02; C12N 1/12; C12P 7/06

(52) U.S. Cl. CPC C12M 21/02; C12M 23/14; C12M 23/26; C12P 7/065; C12M 23/34; C12M 27/00; C12N 1/12; C12M 23/22

(57) ABSTRACT

Photobioreactors having a vertically-oriented enclosure with a thin panel shape, systems providing mixing in an array of photobioreactors, and methods for using the same to culture productive organisms to accumulate biomass and/or make biofuels or other chemical products.

### CROSS-REFERENCE TO RELATED APPLICATIONS

This application is a continuation of International Application No. PCT/US2016/068153, filed Dec. 21, 2016, which claims the benefit of U.S. Provisional Application No. 62/270,458, filed Dec. 21, 2015, the disclosures of which are incorporated by reference.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND

The possibility of using algae for the production of fuel and chemicals has attracted the interest of researchers, government and business for many years. Efforts to commercialize the production of fuel from algae using closed photobioreactors have brought to light problems that must be solved to improve the performance of photobioreactors and make the production of biofuels practical.

Various approaches to photobioreactors are the subject of, for example, U.S. Pat. App. Pub. No. 2011/0151507; U.S. Pat. No. 8,304,209; WO/2005/121309; WO/2010/068288; U.S. Pat. No. 5,534,417; WO/2007/098150; WO/2013/133481; U.S. Pat. App. Pub. No. 2008/0160591; U.S. Pat. App. Pub. No. 2010/0285575; U.S. Pat. App. Pub. No. 2011/0306121; U.S. Pat. App. Pub. No. 2010/0032851; U.S. Pat. App. Pub. No. 2007/0289206; U.S. Pat. App. Pub. No. 2012/0301563; U.S. Pat. App. Pub. No. 2010/0028976; WO/2009/040383; and Christenson et al., “Production and harvesting of microalgae for wastewater treatment, biofuels, and bioproducts”, Biotechnology Advances 29 (2011) 686-702.

An ongoing need exists for photobioreactors and photobioreactor systems offering improved performance and processing attributes.

### SUMMARY

An object of the present invention is a photobioreactor that supports environmental conditions in which productive organisms in a liquid culture inside the enclosure accumulate biomass and/or make biofuels or other chemical products of interest through photosynthesis. A photobioreactor of the present invention may comprise a panel-shaped enclosure with a height that is oriented substantially vertically and further comprises structural features such as point seams that advantageously enhance structural integrity of the enclosure and maintain uniform thickness of the enclosure while minimally impeding flow and mixing of fluids within the enclosure, as well as structural features such as sloped top and bottom edges that facilitate filling and draining of the enclosure. According to the present invention, as the thickness of the enclosure becomes more uniform, the ratio of illuminated liquid culture surface area to liquid culture volume increases, which increases volumetric productivity and product concentration in the liquid culture, thereby reducing energy costs to refine the product. Further according to the present invention, structural features such as sloped top and bottom edges improve operational capabilities of the photobioreactor system.

An aspect of the present invention is directed to a photobioreactor for culturing productive organisms comprising an enclosure comprising a flexible film seamed in a pattern comprising an edge, a plurality of linear seams spaced apart and disposed at a distance from the edge, and a first plurality of point seams spaced apart and disposed at a distance from the plurality of linear seams, wherein the plurality of linear seams is disposed between the edge and the first plurality of point seams, further wherein at least one portion of the flexible film is translucent.

An additional aspect of the present invention is directed to a photobioreactor wherein the spacing between adjacent linear seams in the plurality of linear seams is about 0.75 inches, further wherein the distance from the plurality of linear seams to the edge is about 2.5 inches, further wherein the spacing between adjacent point seams in the first plurality of point seams is about 2 inches, further wherein the distance from the first plurality of point seams to the plurality of linear seams is from about 1.38 inches to about 2.56 inches.

An additional aspect of the present invention is directed to a photobioreactor wherein the pattern further comprises a second plurality of point seams spaced apart and a third plurality of point seams spaced apart, further wherein the second plurality of point seams is disposed at a distance from the first plurality of point seams, further wherein the third plurality of point seams is disposed at a distance from the second plurality of point seams, further wherein the ratio of the distance between the second plurality of point seams and the third plurality of point seams to the spacing between adjacent point seams in the second plurality of point seams is from about 0.125 to about 0.7.

An additional aspect of the present invention is directed to a photobioreactor wherein the edge, the plurality of linear seams, the first plurality of point seams, the second plurality of point seams, and the third plurality of point seams are disposed substantially parallel.

An additional aspect of the present invention is directed to a photobioreactor for culturing productive organisms comprising an enclosure comprising a flexible film comprising seams in a pattern shown in FIG. 3, wherein at least one portion of the flexible film is translucent.

A further object of the present invention is a photobioreactor system comprising at least one photobioreactor enclosure oriented substantially vertically wherein the seams are configured to maintain substantially uniform thickness of the at least one photobioreactor enclosure, further comprising orifices capable of permitting fluid flows into and out of the at least one photobioreactor enclosure; a first header and a second header disposed below, and in fluid communication with, the at least one photobioreactor enclosure; a third header and a fourth header disposed above, and in fluid communication with, the at least one photobioreactor enclosure; a conduit loop in fluid communication with the first, second and third headers and with a gas supply; and a gas diffuser disposed in a lower portion of the at least one photobioreactor enclosure, adapted to add gas into the at least one photobioreactor enclosure and in fluid communication with the fourth header.

A further object of the present invention is a method of culturing productive organisms comprising the steps of inoculating a photobioreactor with a culture of productive organisms; exposing the culture of productive organisms to photosynthetically active radiation; adding liquid to the at least one photobioreactor enclosure through the first header; removing liquid from the at least one photobioreactor enclosure through the second header; adding gas to the at least one photobioreactor enclosure through the fourth header and the gas diffuser, causing circulation of liquid and gas in the photobioreactor system; removing gas from the at least one photobioreactor enclosure through the third header; and adding gas from the gas supply to the conduit loop, causing circulation of liquid and gas in the photobioreactor system.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE SEVERAL VIEWS OF THE DRAWING

Embodiments of the invention will be described below with reference to the following figures.

FIG. 1 shows a plan view of a general structure of a photobioreactor design.

FIG. 2A shows a sectional view of a photobioreactor design.

FIG. 2B shows a sectional view of a photobioreactor design of the present invention.

FIG. 3 shows a plan view of a photobioreactor design of the present invention.

FIG. 4 shows a sectional plan view of a photobioreactor design of the present invention.

FIG. 5 shows a sectional plan view of a photobioreactor design of the present invention.

FIG. 6 shows a sectional plan view of a photobioreactor design of the present invention.

FIG. 7 shows a sectional plan view of a photobioreactor design of the present invention.

FIG. 8 shows a sectional plan view of a photobioreactor design of the present invention.

FIG. 9 shows a sectional plan view of a photobioreactor design of the present invention.

FIG. 10 shows a sectional plan view of a photobioreactor design of the present invention.

FIG. 11 shows a sectional plan view of a photobioreactor design and headers of the present invention.

FIG. 12 shows a plan view of a photobioreactor design of the present invention.

FIG. 13 shows a plan view of a photobioreactor design of the present invention.

FIG. 14 shows a plan view of a photobioreactor design of the present invention.

FIG. 15 shows a plan view of a gas diffuser of a photobioreactor system of the present invention.

FIG. 16 shows a top perspective view of a photobioreactor system of the present invention.

FIGS. 17A and 17B show plan and perspective views of a conduit loop of a photobioreactor system of the present invention.

### DETAILED DESCRIPTION

The present invention relates to photobioreactors, systems and methods for using a wild-type or metabolically enhanced microorganism in a photobioreactor of the present invention to develop biomass or make chemical products through photosynthesis.

As used herein, the term “productive organism” means a microorganism that carries out photosynthesis and accumulates biomass in wild-type form, and also is competent to be metabolically enhanced, such that the enhanced microorganism metabolizes an intermediate that is made through photosynthesis and converts the intermediate into a chemical product of interest. The cell can be a prokaryotic or a eukaryotic cell. The term is intended to include progeny of the cell originally metabolically enhanced. Non-limiting examples of productive organisms within the meaning of the present invention are Cyanobacterium, Synechococcus, Synechocystis and Chlorogloeopsis species. In some embodiments, the cell is a prokaryotic cell, e.g., a cyanobacterial cell. One of ordinary skill in the art will recognize that other productive organisms are within the scope of the present invention.

As used herein, the term “metabolically enhanced” means any change in the endogenous genome of a productive organism or to the addition of non-endogenous genetic code to a productive organism, e.g., the introduction of a heterologous gene. More specifically, such changes are made by the hand of man through the use of recombinant DNA technology or mutagenesis. The changes can involve protein coding sequences or non-protein coding sequences.

For commercial-scale operations employing a photobioreactor system of the present invention, it is preferable to use a productive organism that is metabolically enhanced in a manner such that the productive organism produces only, or predominantly, one target product. Metabolic enhancements that are effective to achieve this result may be implemented by using known genetic engineering techniques to introduce selected genes into cells of the productive organism. Examples of metabolically enhanced microorganisms that produce ethanol through conducting photosynthesis are disclosed in U.S. Pat. No. 6,306,639 to Woods et al. titled “Genetically Modified Cyanobacteria For The Production Of Ethanol, The Constructs And Method Thereof.”

As used herein, the term “photosynthesis” means a process used by organisms to convert light energy captured from the sun into chemical energy that can be used to fuel the organism's activities. In productive organisms of the present invention, light energy and carbon are further converted into biomass and/or chemical products such as biofuels.

As used herein, the term “biomass” means organic matter derived from living, or recently living organisms, which may be used in the production of, for example, biocrude, biofertilizer and biostimulants.

As used herein, the term “chemical product” means an organic compound made by a productive organism through photosynthesis. Non-limiting examples of chemical products are biofuels and colorants.

As used herein, the term “biofuel” means a type of fuel that derives energy from biological carbon fixation. Biofuels include fuels derived from biomass conversion or from cell metabolism, as well as solid biomass, liquid fuels and various biogases. Biofuels may be produced by the action of enhanced productive organisms through photosynthesis. A non-limiting example of a biofuel is ethanol.

As used herein, the term “colorant” means any substance that imparts color. Non-limiting examples of colorants are phycocyanin and phycoerythrin.

As used herein, the term “photobioreactor” means a device or system used to support a biologically active environment for the cultivation of productive organisms in water or a liquid medium that contains nutrients and other growth-promoting constituents. A photobioreactor of the present invention may be constructed of translucent materials that permit penetration of light, or may otherwise incorporate a light source to provide photonic energy input for an aqueous culture of photosynthetic microorganisms contained therein. A photobioreactor of the present invention may be closed or semi-closed against the exchange of gases and contaminants with the outside environment. A photobioreactor of the present invention may be constructed from a flexible film or from a rigid thermoplastic.

As used herein, the term “translucent” means allowing light to pass through, with or without scattering of photons.

As used herein, the term “flexible film” means a continuous polymeric material or coating that is not structurally self-supporting, and preferably is at least partially translucent. Non-limiting examples of materials that can be used in flexible films suitable for use with the present invention are polyolefins, polyesters and vinyl copolymers thereof, including polyethylene, polypropylene, nylon, ethylene vinyl acetate and polyvinyl chloride.

As used herein, the term “thermoplastic” means a material consisting of a synthetic or semi-synthetic organic polymer that becomes pliable or malleable above a specific temperature, such that it can be molded into solid objects, and solidifies upon cooling. Non-limiting examples of thermoplastics suitable for use with the present invention are polycarbonate, polymethyl methacrylate, polyvinyl chloride and polyesters such as polyethylene terephthalate. Thermoplastics suitable for use with the present invention preferably are semi-rigid or rigid and at least partially translucent.

As used herein, the term “point seam” means discrete portions of flexible film or thermoplastic bonded together through radio frequency welding, ultrasonic welding, impulse welding, thermoforming or any other suitable technique, the discrete bonded film portions having the shape of, for example, a circle, oval, polygon with rounded corners or irregular form with rounded corners.

As used herein, the term “header” means a chamber, such as a length of pipe, to which the ends of a number of conduits, such as tubes or pipes typically of smaller diameter than the header, are connected so that liquids and/or gases are distributed from the header among the conduits (supply) or are collected in the header from the conduits (exhaust).

As used herein, the term “sparging” means a process whereby a gas is bubbled through a fluid.

As used herein, the term “gas diffuser” means a mechanical device that sparges or bubbles a gas to control its velocity and enhance its mixing into a surrounding fluid.

As used herein, the term “medium” means a liquid or gel designed to support the growth of microorganisms.

As used herein, the term “in fluid communication with” means a connection that permits the passage of liquids or gases between the recited components.

As used herein, the term “about” means approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) within a confidence interval of 90% or 95%.

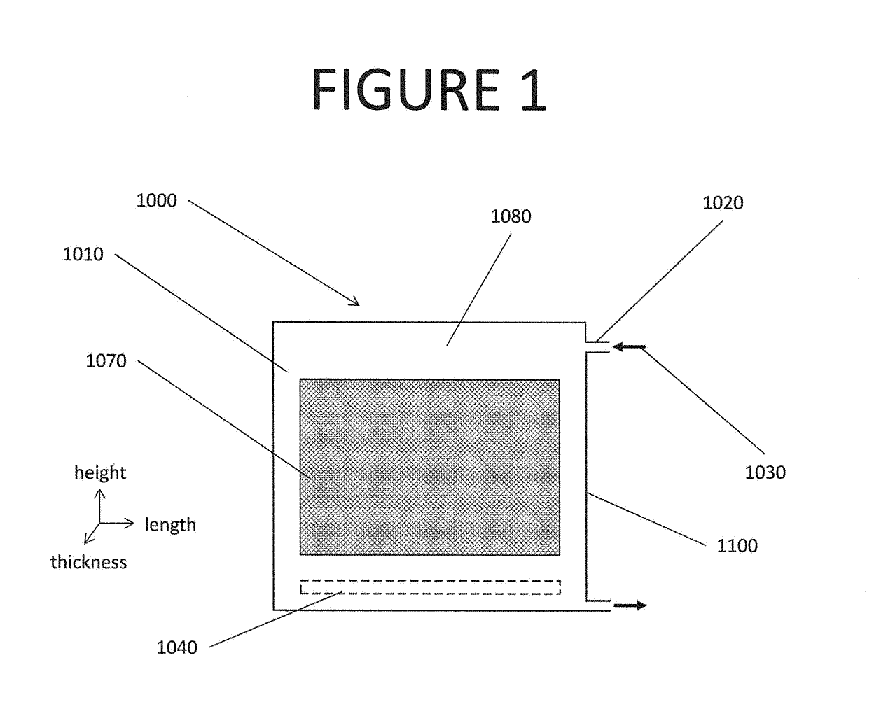

FIG. 1 shows a generic embodiment of a photobioreactor system 1000 of the present invention that comprises a photobioreactor enclosure 1010. In some embodiments, the photobioreactor enclosure 1010 is capable of housing a liquid culture of productive organisms 1070 that fills the photobioreactor enclosure 1010 partially or fully, and a headspace 1080 filled with gas above the surface of the liquid in any portion of the photobioreactor enclosure 1010 that is not filled with liquid.

The shape and dimensions of the photobioreactor enclosure 1010 may be selected to maximize the ratio of surface area to volume, in order to maximize light exposure and distribution over the liquid culture of productive organisms 1070. In some embodiments, the shape of the photobioreactor enclosure 1010 is a thin panel, with a height, a length, and a cross-sectional thickness that is less than either of the height or the length.

The photobioreactor enclosure 1010 may be configured to accommodate entering and exiting flows 1030 of gas and liquid through orifices or ports 1020 in the photobioreactor enclosure 1010.

The photobioreactor system 1000 may comprise a gas diffuser 1040 that is configured to bubble gas into the contents of the photobioreactor enclosure 1010.

The photobioreactor enclosure 1010 may comprise flexible film that forms two opposing walls 1060. In some embodiments, the flexible film is polyethylene about 0.0075 inch to about 0.0085 inch thick. The opposing walls 1060 may be formed by, for example, two separate sheets of flexible film, one folded sheet of flexible film, or a tube or annular cylinder of flexible film.

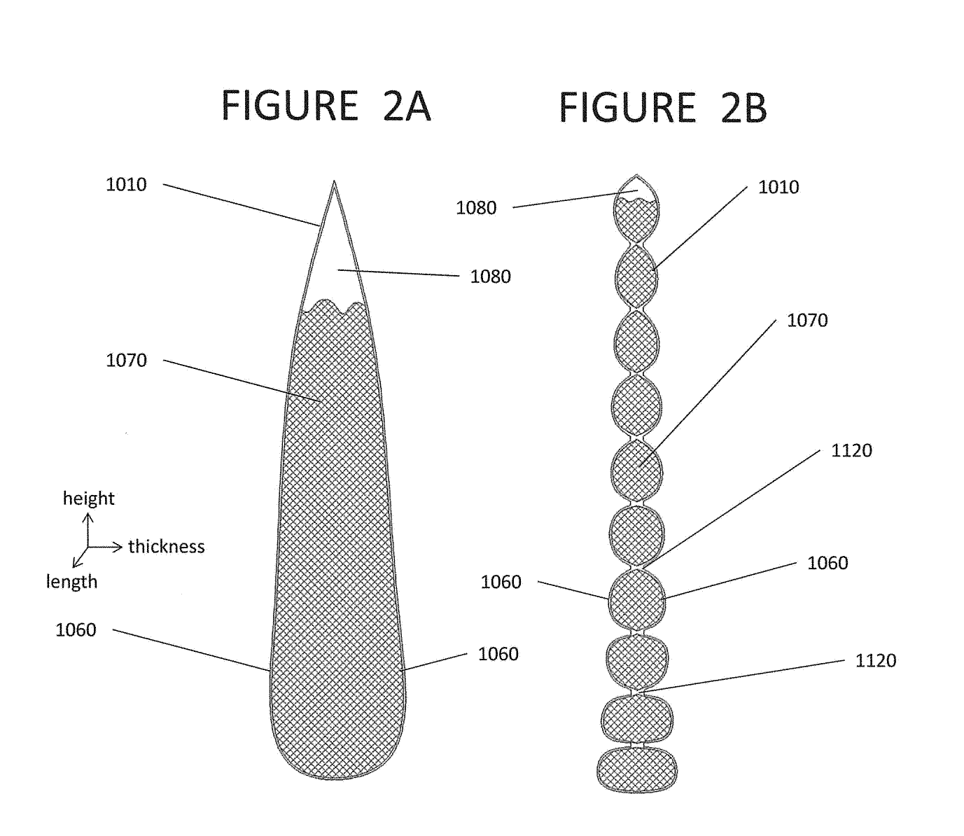

As shown in FIGS. 2A and 2B, liquids and/or gases contained in the photobioreactor enclosure 1010 exert outward pressure that tends to induce curvature of the opposing walls 1060. The photobioreactor enclosure 1010 may further comprise structural features configured to enhance structural integrity and maintain more uniform cross-sectional thickness from top to bottom of the photobioreactor enclosure 1010.

In some embodiments, opposing walls 1060 comprising flexible film are bonded together through radio frequency welding, ultrasonic welding, impulse welding, thermoforming or any other suitable technique to create bonded seams at the perimeter 1100 of the photobioreactor enclosure 1010. Exemplary properties of a suitable flexible film for use in a photobioreactor enclosure 1010 of the present invention are translucency, tolerance to UV radiation, low cost, light weight and acceptable durability.

In some embodiments, at least one opposing wall 1060 of the photobioreactor enclosure 1010 is translucent or transparent for the purpose of allowing exposure of the liquid culture of productive organisms 1070 in the internal void volume to light from the sun or another source that provides photosynthetically active radiation having wavelengths from 400 to 700 nanometers. The liquid culture of productive organisms 1070 utilizes the light to accumulate biomass and/or make chemical products of interest through photosynthesis.

The dimensions of the photobioreactor enclosure 1010 can be selected to optimize properties such as photosynthetic efficiency of the liquid culture of productive organisms 1070 contained in the photobioreactor enclosure 1010 and productivity per unit cost of capital and operation, as determined by considerations of light exposure and dilution, dynamics of mixing of liquids and gases in the photobioreactor enclosure 1010 and costs of build materials, energy inputs and supporting structures, for example. In some embodiments, the height of the photobioreactor enclosure 1010 ranges from about 2 feet to about 7 feet, and more preferably from about 4 feet to about 5 feet. In some embodiments, the length of the photobioreactor enclosure 1010 ranges from about 10 feet to about 30 feet, and more preferably about 20 feet. In some embodiments, the maximum cross-sectional average thickness of the photobioreactor enclosure 1010 ranges from about 1 centimeter to about 3 centimeters, more preferably from about 1.5 centimeters to about 2 centimeters. The orientation of the height of the photobioreactor enclosure 1010 is substantially vertical, and the orientation of the length and thickness of the photobioreactor enclosure 1010 are substantially horizontal and orthogonal.

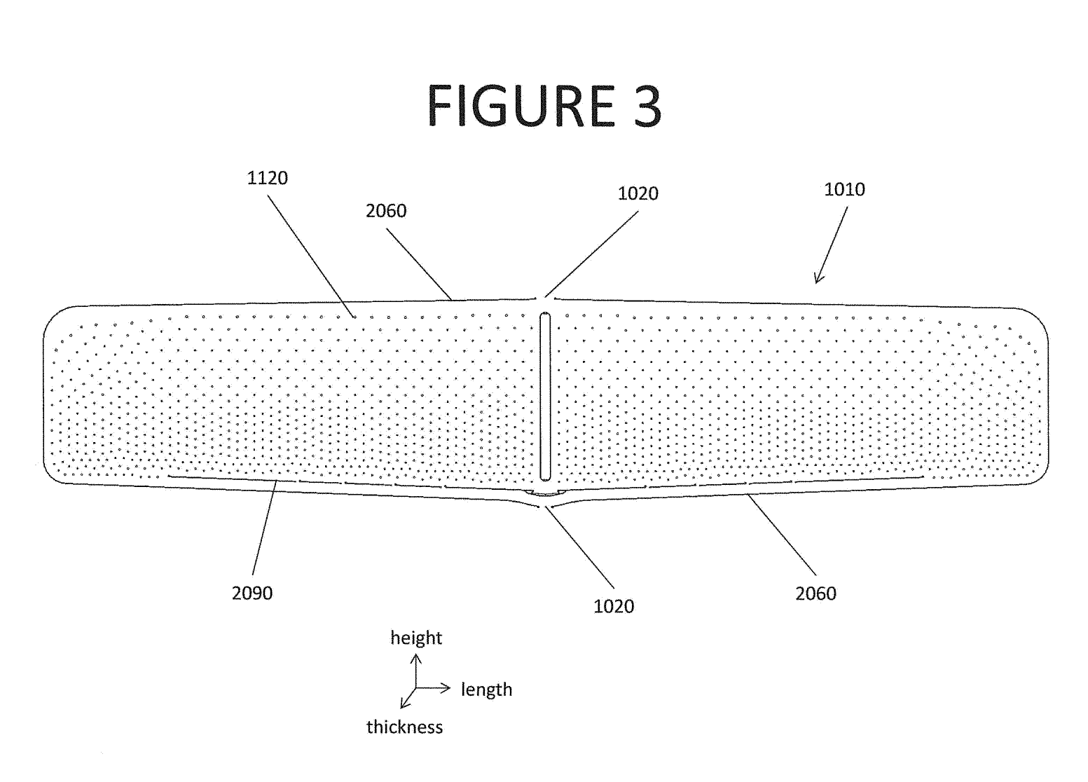

FIG. 3 shows a seaming pattern, drawn to scale, for an embodiment of a photobioreactor enclosure 1010 configured as a thin panel with an internal void volume. In some embodiments, the height of the photobioreactor enclosure 1010 is oriented more or less vertically. In some embodiments, the photobioreactor enclosure 1010 is constructed of flexible film and comprises linear or curvilinear seams that define the perimeter of the photobioreactor enclosure 1010 and point seams 1120 dispersed within the void volume of the photobioreactor enclosure 1010. The point seams 1120 may be formed by, for example, bonding together discrete portions of the opposing walls 1060 comprising the flexible film so as to create bonded seams. The diameter or width of the point seams 1120 is in the plane defined by the length and width of the photobioreactor enclosure 1010.

In some embodiments, point seams 1120 enhance structural integrity of the photobioreactor enclosure 1010 by dispersing fluid pressure exerted on the opposing walls 1060, and also minimally impede flow and mixing of liquids and gases in the photobioreactor enclosure 1010.

Pressure head from liquid completely or partially filling a photobioreactor enclosure 1010 constructed of flexible film tends to displace the opposing walls 1060 apart. In a photobioreactor enclosure 1010 without point seams 1120, as shown in FIG. 2A, the pressure head creates a bulge at or near the bottom of the photobioreactor enclosure 1010, such that the thickness of a lower portion of the photobioreactor enclosure 1010 is greater than the thickness of an upper portion of the photobioreactor enclosure 1010. In a photobioreactor enclosure 1010 comprising point seams 1120, as shown in FIG. 2B, the point seams 1120 increase the surface area and points of attachment between the opposing walls 1060 and constrain the displacement of the opposing walls 1060, thereby reducing bulging. In some embodiments, the thickness of a flexible film photobioreactor enclosure 1010 comprising point seams 1120 and completely or partially filled with liquid is substantially more uniform than the thickness of a flexible film photobioreactor enclosure 1010 without point seams 1120, as shown in FIGS. 2A and 2B.

According to the present invention, as the thickness of a photobioreactor enclosure 1010 containing a liquid culture of productive organisms 1070 becomes more uniform and bulging diminishes, the ratio of illuminated liquid culture surface area to liquid culture volume increases. Consequently, volumetric productivity of the liquid culture of productive organisms 1070 increases, which increases product concentration in the liquid culture and advantageously reduces costs to refine product made by the liquid culture. In some embodiments of a photobioreactor enclosure 1010 containing a static volume of liquid culture, the ratio of illuminated liquid culture surface area to liquid culture volume and the volumetric productivity are higher with point seams 1120 disposed in the photobioreactor enclosure 1010, as in FIG. 2B, than without point seams 1120, as in FIG. 2A.

Under pressure head from liquid filling a photobioreactor enclosure 1010, opposing walls 1060 comprising flexible film may fold or crease between point seams 1120 and contact each other, thereby restricting fluid flow in the photobioreactor enclosure 1010. In some embodiments, the photobioreactor enclosure 1010 comprises point seams 1120 that are spaced apart so as to distribute mechanical stresses in a manner that minimizes or substantially eliminates formation of folds or creases in the walls 1060.

In some embodiments, point seam 1120 diameter or width is about 0.75 inches or smaller. In some embodiments, point seam 1120 diameter or width is from about 0.375 to about 0.75 inches. In some embodiments, the shape of the point seam 1120 is a circle, oval, polygon with rounded corners or irregular form with rounded corners. According to the present invention, rounded or large-angle corners in the point seam 1120 shape are beneficial to fabrication of the point seam 1120 and resistance of the point seam 1120 to tearing and creating a leak in the photobioreactor enclosure 1010. In some embodiments, a point seam 1120 has any discrete shape and size that are suitable to enhance structural integrity of the photobioreactor enclosure 1010 while minimally impeding fluid flow and mixing therein.

Mechanical stress incident on a first point seam 1120 located in a first region of a photobioreactor enclosure 1010 may differ substantially from mechanical stress incident on a second point seam 1120 located in a distinct second region of the photobioreactor enclosure 1010, due to differences in, for example, pressure head and mass of the photobioreactor enclosure 1010 below the point seam 1120. In some embodiments, for the purpose of maintaining uniform thickness of the photobioreactor enclosure 1010, spacing between point seams 1120 in different regions of a photobioreactor enclosure 1010 is varied to compensate for varying mechanical stresses incident on the point seams 1120 in different regions. In some embodiments, vertical or horizontal spacing between point seams 1120 in different regions of a photobioreactor enclosure 1010 varies from about 0.5 inches to about 6 inches.

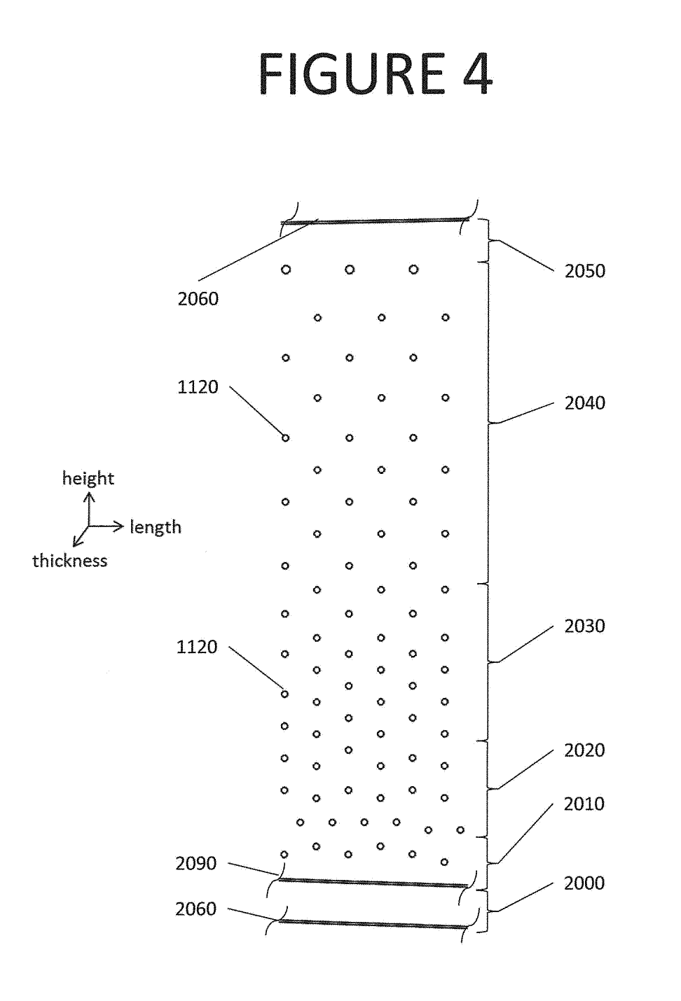

FIGS. 4-10 illustrate arrangements of point seams 1120 and other structural features in the exemplary vertical photobioreactor enclosure 1010 shown in FIG. 3 made of flexible film, drawn to scale in each Figure.

FIG. 4 shows a sectional view of horizontal regions 2000, 2010, 2020, 2030, 2040, 2050 of the photobioreactor enclosure 1010 shown in FIG. 3, increasing in vertical height from region 2000 to region 2050. A gas diffuser would be positioned in region 2010. A gas headspace region would be found in region 2050.

Each region experiences varying mechanical stress caused by aggregate weight and pressure head applied to a region. Aggregate weight of the photobioreactor enclosure 1010 and liquid suspended below a region decreases from higher regions, such as region 2050, to lower regions, such as region 2010. Pressure head from liquid filling the photobioreactor enclosure 1010 increases from higher regions, such as region 2050, to lower regions, such as region 2010. In a photobioreactor enclosure 1010 filled with about 100 liters of liquid to an average fill height of about 36 inches, pressure head in region 2000 is about 34 to about 36 inches of water, pressure head in region 2010 is about 31 to about 34 inches of water, pressure head in region 2020 is about 25 to about 31 inches of water, pressure head in region 2030 is about 17 to about 25 inches of water, and pressure head in region 2040 is about 0 to about 17 inches of water. The gas headspace region in region 2050 can be inflated to a pressure of, for example, about 10 inches of water, which would increase pressure in the other regions equally.

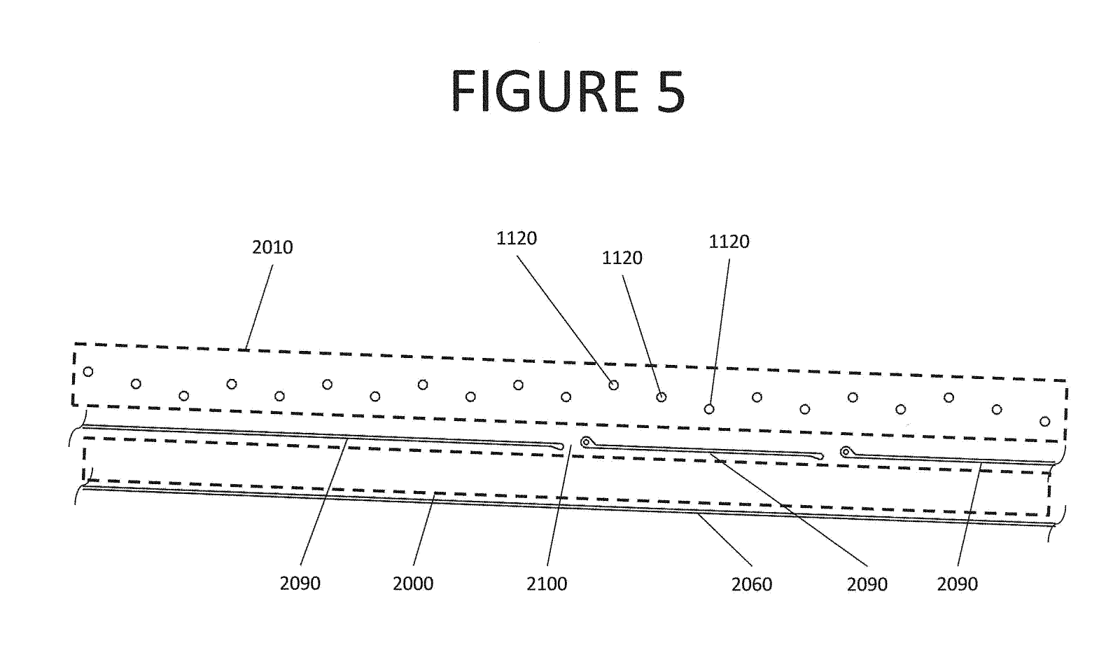

FIG. 5 shows region 2000 and arrangement of the point seams 1120 in region 2010. A weld along the perimeter of the photobioreactor enclosure 1010 forms the bottom, top and side edges 2060 of the photobioreactor enclosure 1010. The bottom and top edges 2060 are sloped from horizontal.

A series of seams 2090 in alignment is positioned about 2.5 inches above the bottom edge 2060. In some embodiments, the seams 2090 are linear seams that are substantially longitudinally aligned. In some embodiments, the seams 2090 are point seams arranged substantially linearly. Each gap 2100 in the series of seams 2090 allows bubbles to escape from the channel bounded by the bottom edge 2060 and the series of seams 2090, in order to prevent blockage of the channel by bubbles. In some embodiments, the gap 2100 is about 0.75 inches.

Point seams 1120 are positioned between about 1.38 and 2.56 inches above the series of seams 2090, and spaced laterally about 2 inches between each point seam 1120. The arrangement of point seams 1120 in region 2010 provides a sufficiently large void volume in the photobioreactor enclosure 1010 to allow insertion of a gas diffuser tube (not shown) between point seams 1120 and the series of seams 2090 and to enable full draining of liquids through the channel bounded by the bottom edge 2060 and the series of seams 2090.

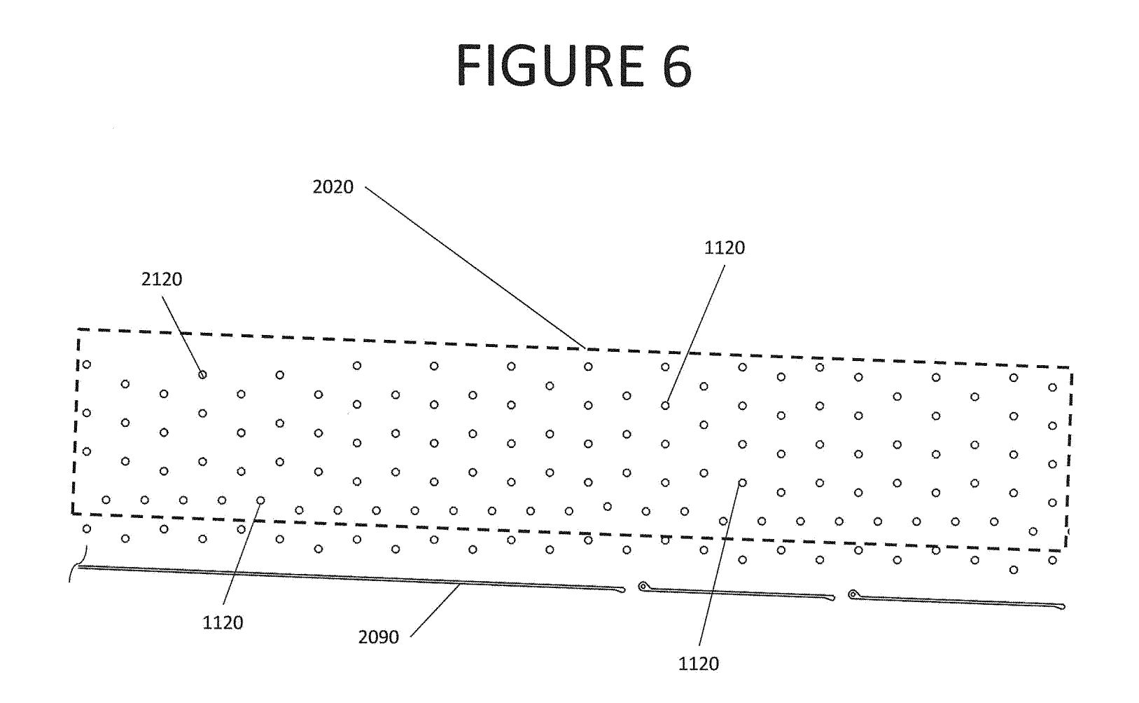

FIG. 6 shows the arrangement of point seams 1120 in region 2020 of the photobioreactor enclosure 1010. Point seams 1120 are spaced apart laterally about 2 inches.

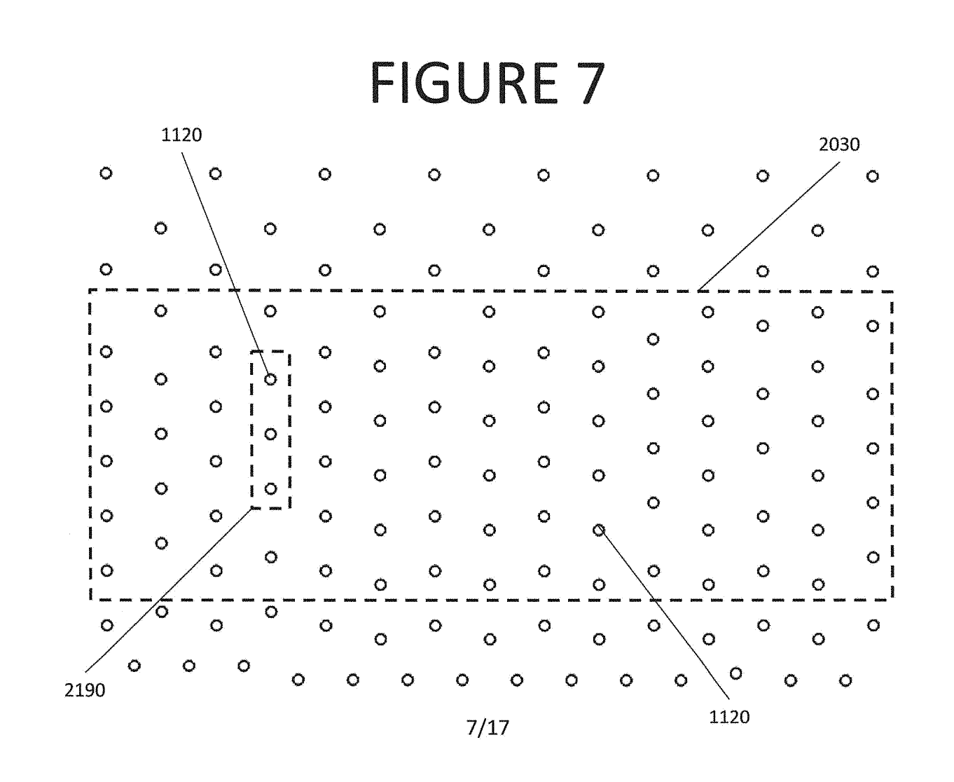

FIG. 7 shows the arrangement of point seams 1120 in region 2030 of the photobioreactor enclosure 1010. Point seams 1120 in subregion 2190 are spaced apart vertically about 2 inches.

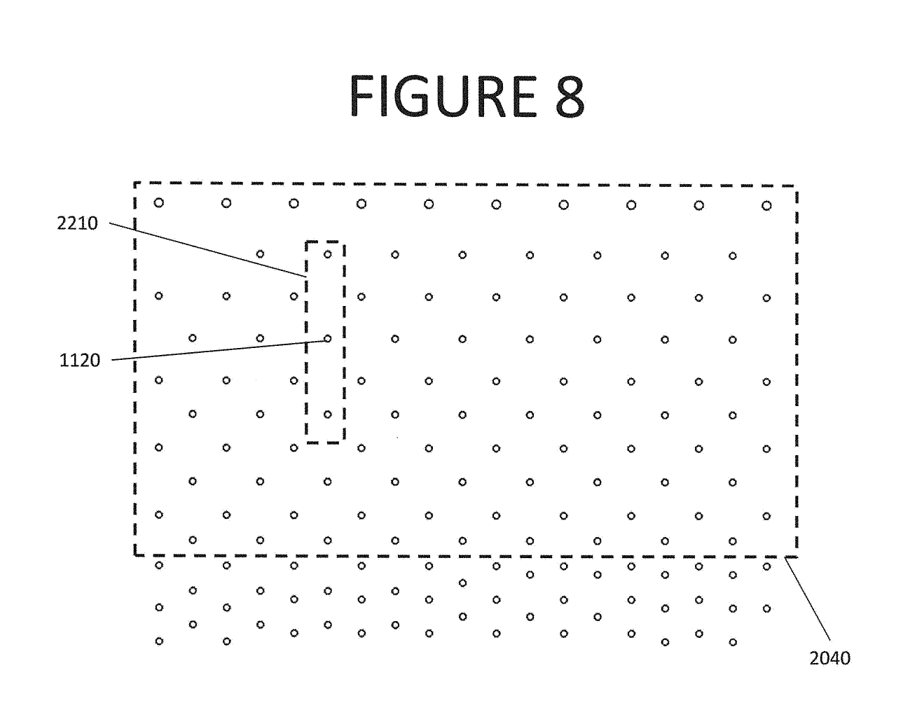

FIG. 8 shows the arrangement of point seams 1120 in region 2040 of the photobioreactor enclosure 1010. Point seams 1120 in subregion 2210 are spaced apart vertically about 4.5 inches.

Diameter of the point seams in FIGS. 3-10 is 0.375 inches, except that diameter of the uppermost point seams that would be covered by liquid in the photobioreactor enclosure is 0.5 inches. Additional point seams in the gas headspace of the photobioreactor enclosure may be 0.5 inches or more in diameter.

Photobioreactor enclosures 1010 of the present invention comprise openings, orifices, or ports 1020, to allow, for example, the addition and removal of liquids and gases to and from the internal void volume of the photobioreactor enclosure 1010 and the insertion of probes for monitoring pH, temperature, oxygen concentration, carbon dioxide concentration, ethanol concentration and other properties within the photobioreactor enclosure 1010.

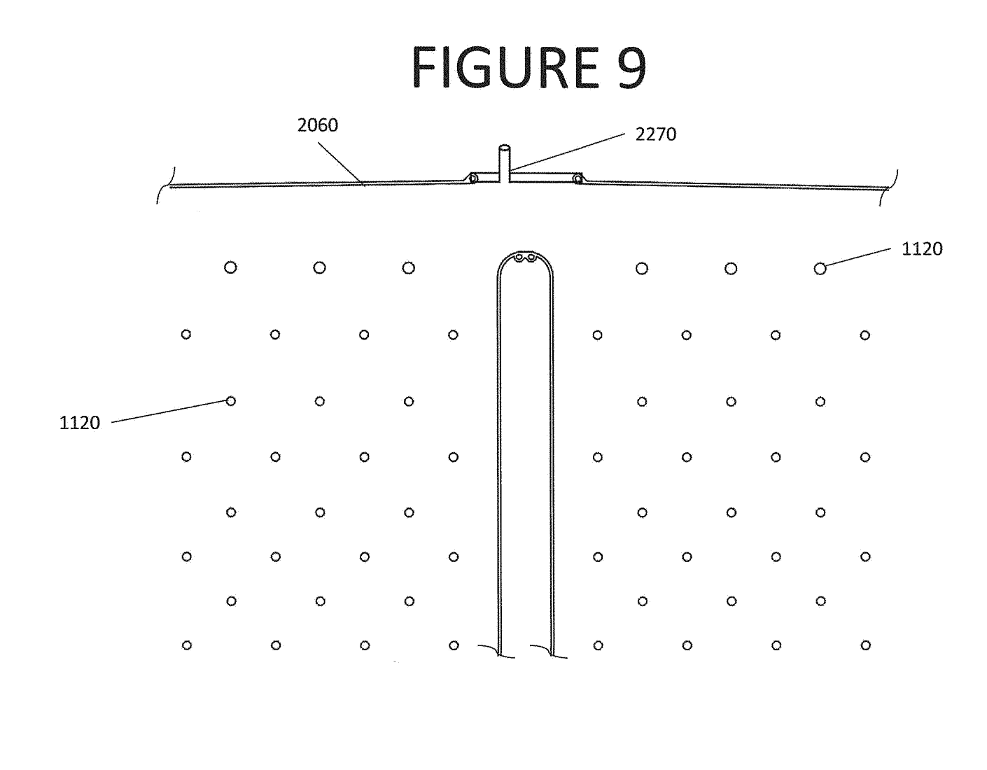

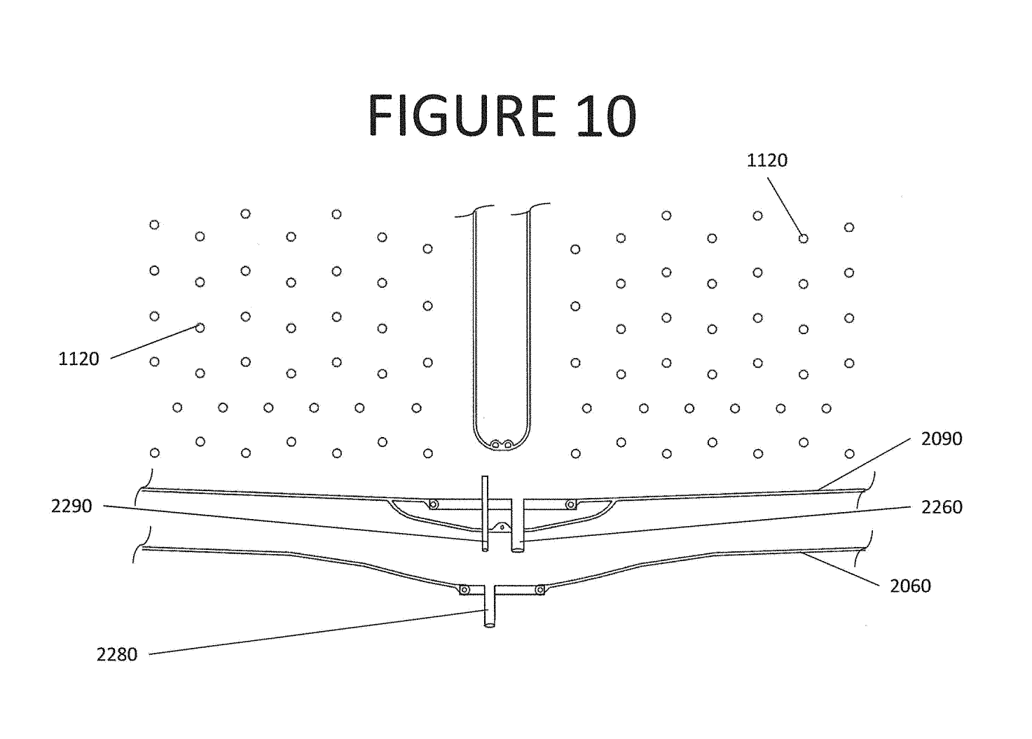

FIGS. 9 and 10 show a gas out orifice 2270 disposed along the top edge 2060 at or near the center of the photobioreactor enclosure 1010, and a liquid in orifice 2260, a liquid out orifice 2280, and a gas in orifice 2290 disposed along the bottom edge 2060 at or near the center of the photobioreactor enclosure 1010. In this configuration, the liquid out orifice 2280 acts as a gravity drain, while rising gas may be vented through the gas out orifice 2270. A gas addition line positioned near or above the top of the photobioreactor enclosure 1010 can be diverted to connect with the gas in orifice 2290 introduce gas to the bottom of the photobioreactor enclosure 1010, so as to bubble gas upward through liquid contained in the photobioreactor enclosure 1010.

An exemplary inner diameter of the liquid and gas in orifices may be about 0.125 inches. An exemplary inner diameter of the liquid and gas out orifices may be about 0.625 inches.

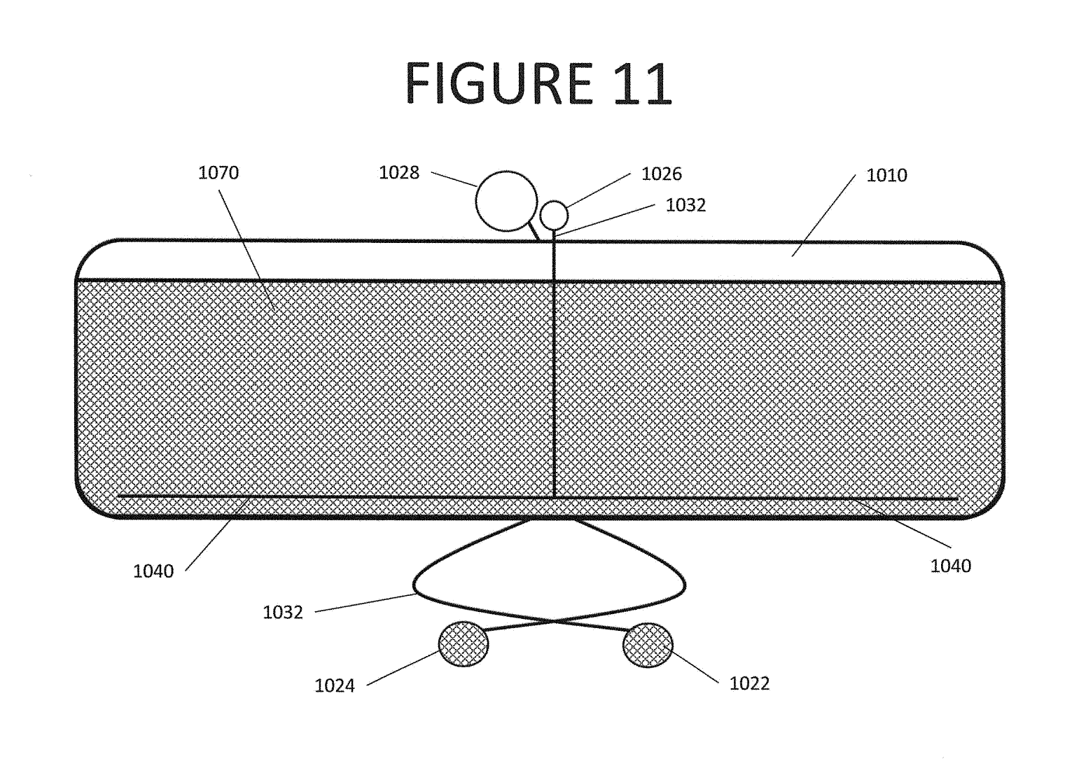

In some embodiments, such as shown in FIG. 11, one or more photobioreactor enclosures 1010 are connected by tubing or piping 1032 to common headers, such as a liquid flow in header 1022, a liquid flow out header 1024, a gas flow in header 1026, and a gas flow out header 1028. In some embodiments, the headers are constructed of thermoplastic, such as polyvinyl chloride.

In some embodiments, a liquid flow out header 1024 is positioned below the bottoms of the photobioreactor enclosures 1010, so that liquid can be drained by gravity feed from the photobioreactor enclosures 1010 to the liquid flow out header 1024.

In some embodiments, a liquid flow in header 1022 is positioned below the bottoms of the photobioreactor enclosures 1010. In some embodiments, the point of connection between the liquid flow in header 1022 and a corresponding orifice 1020 is on the side of the liquid flow in header 1022, and the tubing 1032 that connects the liquid flow in header 1022 with the orifice 1020 is positioned such that no portion of the tubing 1032 dips below the height of the point of connection between the liquid flow in header 1022 and the corresponding orifice 1020. According to the present invention, such a configuration helps eliminate formation of gas bubbles in the tubing 1032 that would otherwise interfere with liquid flow 1030.

In some embodiments, a gas flow in header 1026 is positioned above the tops of the photobioreactor enclosures 1010, or above the height of liquid filling the photobioreactor enclosures 1010, and is connected by tubing or piping 1032 to a gas diffuser 1040 positioned in the bottom of the photobioreactor enclosure 1010. The elevated position of the gas flow in header 1026 may prevent flooding of the header by liquid in the photobioreactor enclosure 1010, in the event gas flow 1030 through the gas diffuser 1040 is interrupted.

In some embodiments, a gas flow out header 1028 is positioned above the tops of the photobioreactor enclosures 1010, or above the height of liquid filling the photobioreactor enclosures 1010. Gas bubbled through the gas diffuser 1040 into the liquid culture is vented through the orifice 1020 near the top of the photobioreactor enclosure 1010 connected to the gas flow out header 1028.

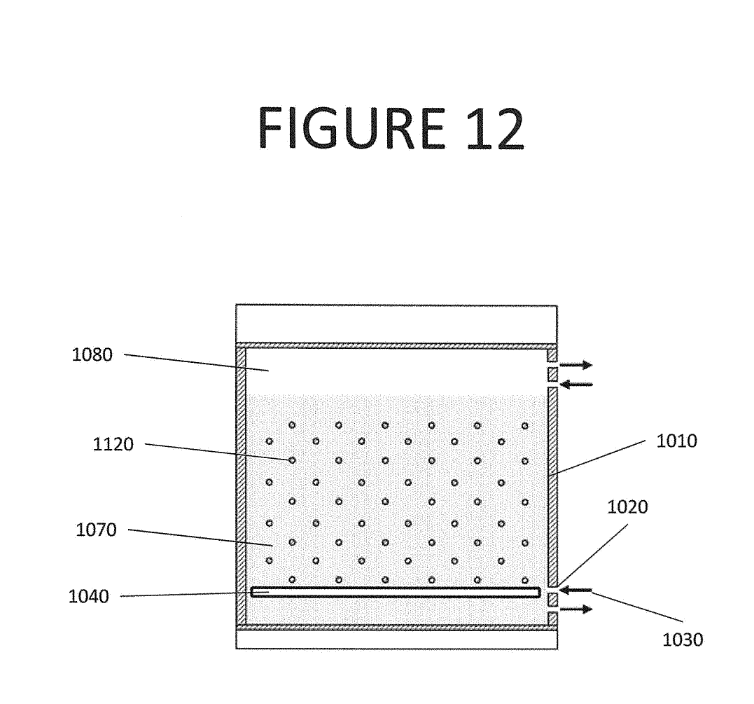

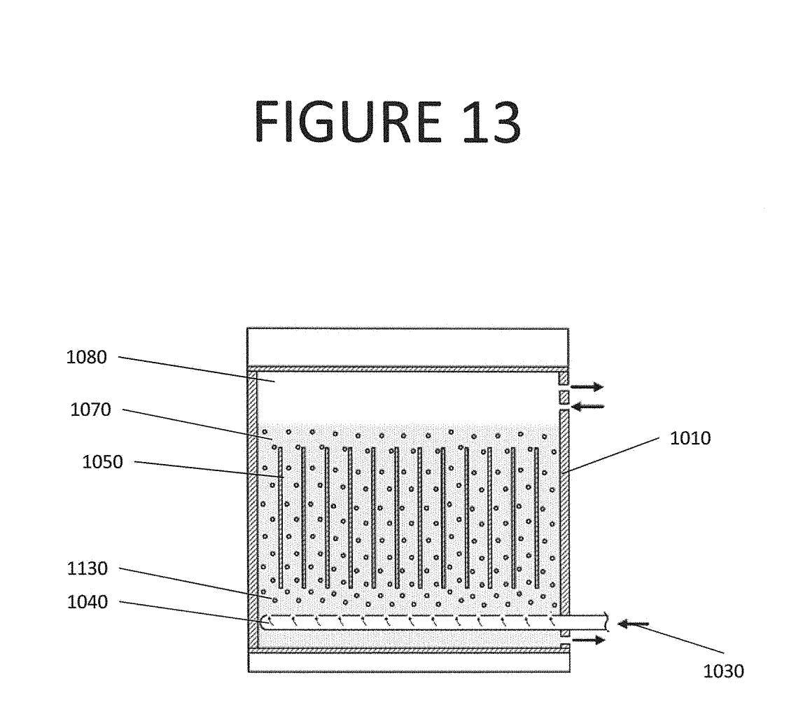

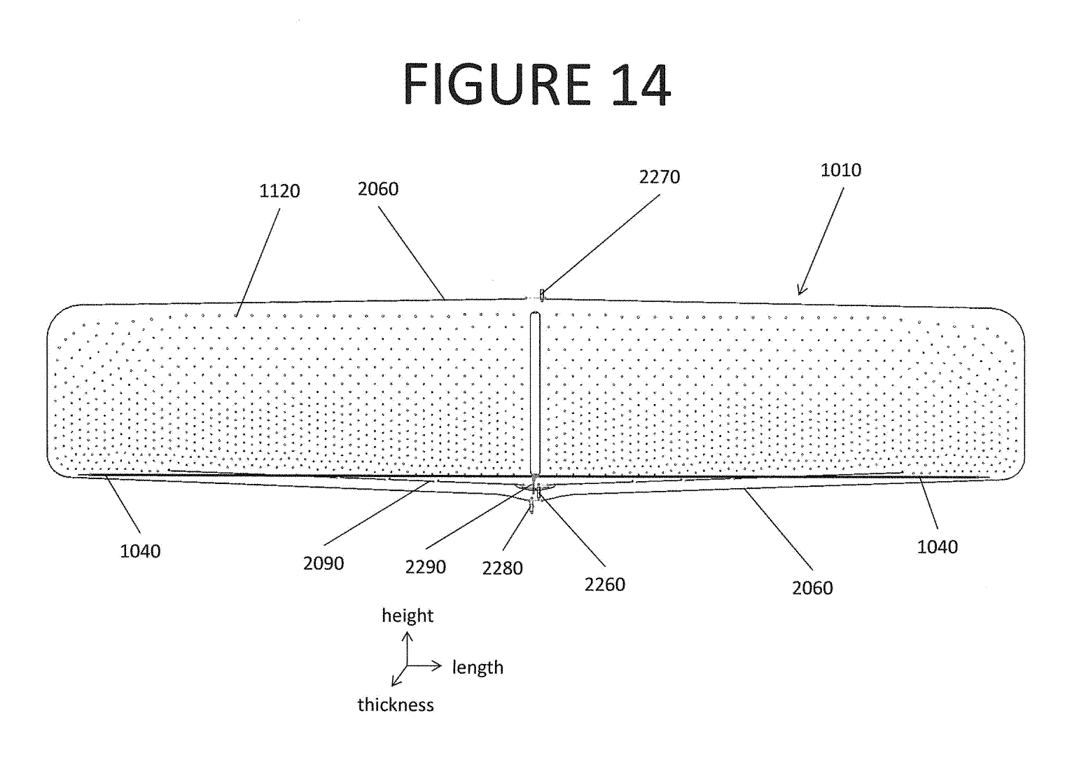

As shown in FIGS. 12-14, in some embodiments, a gas diffuser 1040 is positioned in the bottom of the photobioreactor enclosure 1010 and connected to an orifice 1020 for gas flows 1030 positioned near the bottom of the photobioreactor enclosure 1010. In some embodiments, the gas diffuser 1040 is a tube, or sparging hose, that is perforated partially or completely along its length, with roughly circular, oval or similarly-shaped holes. Gas is bubbled through liquid culture medium 1070 contained in the photobioreactor enclosure 1010 by directing gas flows 1030 through the orifice 1020 and the gas diffuser 1040. The gas escapes through the perforations in the gas diffuser 1040 and forms bubbles 1130 that rise through the culture medium 1070 into the gas headspace 1080, where the gas is expelled though an orifice 1020.

The gas diffuser 1040 may be made of natural rubber, ethylene propylene diene monomer, nitrile rubber, fluoroelastomer rubber, plastic, foam rubber, dense rubber, silicone rubber or any other material that is suitably impermeable to act as a conduit for gas but can be perforated to enable selective escape of the gas. The gas diffuser 1040 preferably is made of a low cost material that is flexible so as to increase its resistance to fouling and provide more uniform bubble 1130 distributions in comparison to rigid diffusers. The material selected for the gas diffuser 1040 preferably is resistant to exposure to sterilizing agents, and minimizes pressure drop along the length of the gas diffuser 1040, but maintains a minimum pressure drop across the gas diffuser 1040 that is substantially greater than the static pressure head variation to which the gas diffuser 1040 is subject because of its variation in depth. Any materials used in construction of the gas diffuser 1040 should be nontoxic or capable of being detoxified. The gas diffuser 1040 is fashioned such that it provides control over perforation aperture size and bubble 1130 diameter.

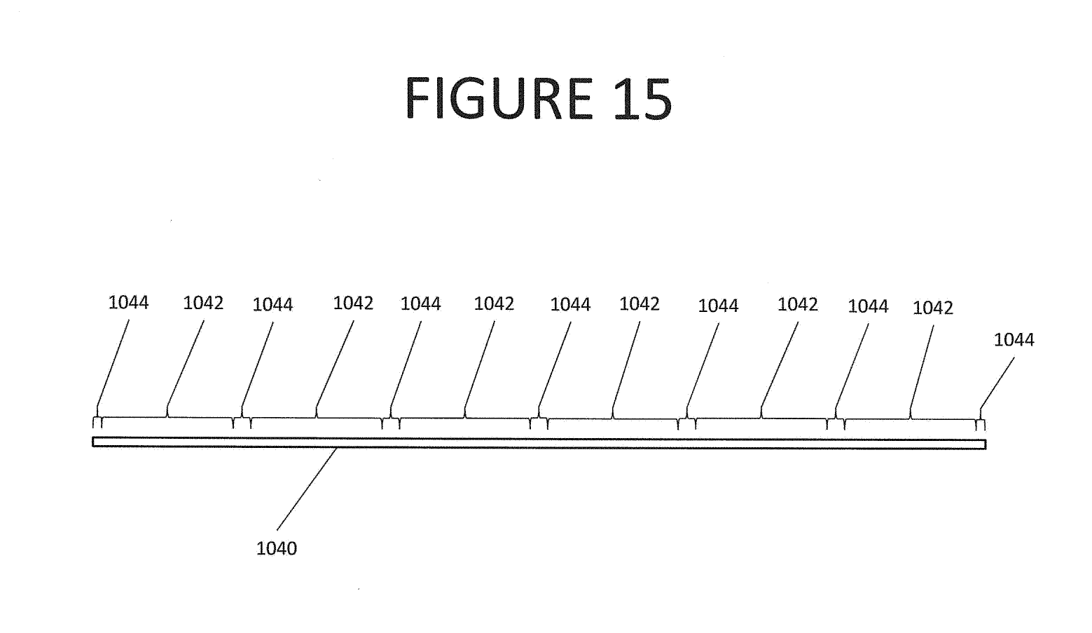

In some embodiments, as illustrated in FIG. 15, a tubular gas diffuser 1040 is configured with repeating perforated sections 1042 and non-perforated sections 1044. Each non-perforated section 1044 is from about 0 to about 5 inches long, preferably from about 1.5 inches to about 5 inches long. Each perforated 1042 section is from about 15 inches long to about the length of the gas diffuser 1040, with from about 1 to about 6 perforations per inch and perforation diameter of from about 0.03 to about 0.08 inches. In some embodiments, total length of the gas diffuser 1040 is from about 105 to about 115 inches, and inner diameter of the gas diffuser 1040 is from about 0.1 to about 0.25 inches to accommodate varying gas pressures and flowrates. Total length of the gas diffuser 1040 may be selected based on length of the photobioreactor enclosure 1010, for example a gas diffuser 1040 length less than or almost as long as the photobioreactor enclosure 1040. In some embodiments, the gas diffuser 1040 comprises ethylene propylene diene monomer.

Gas is bubbled into portions of the liquid culture medium above the perforated sections 1042, while gas is not bubbled into other portions of the liquid culture medium above the non-perforated sections 1044. According to the present invention, the pattern of alternating bubbling and non-bubbling induces mixing throughout the liquid culture medium.

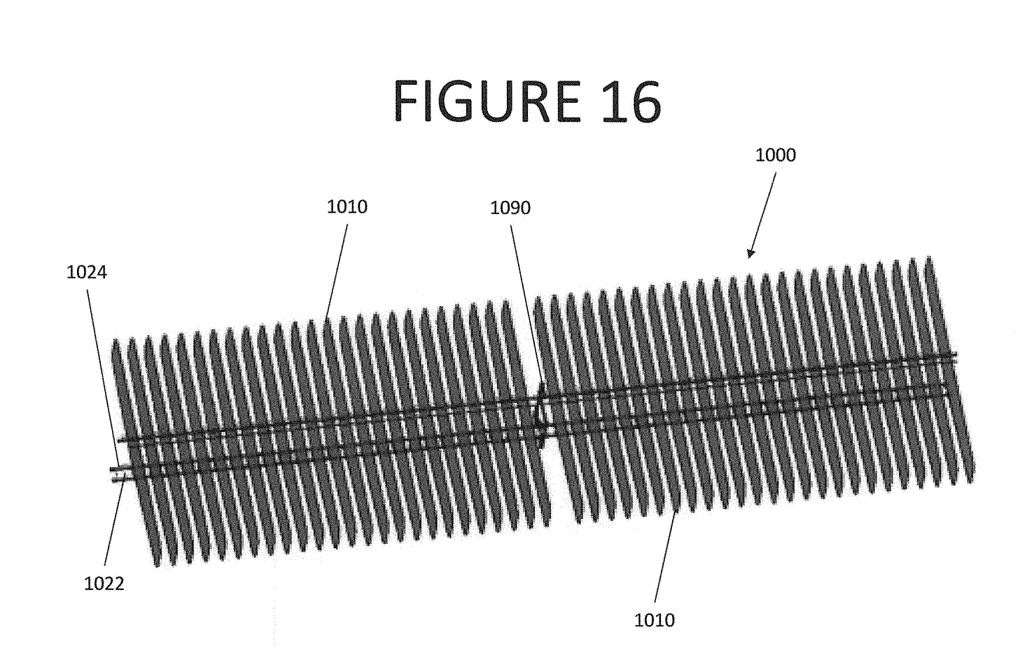

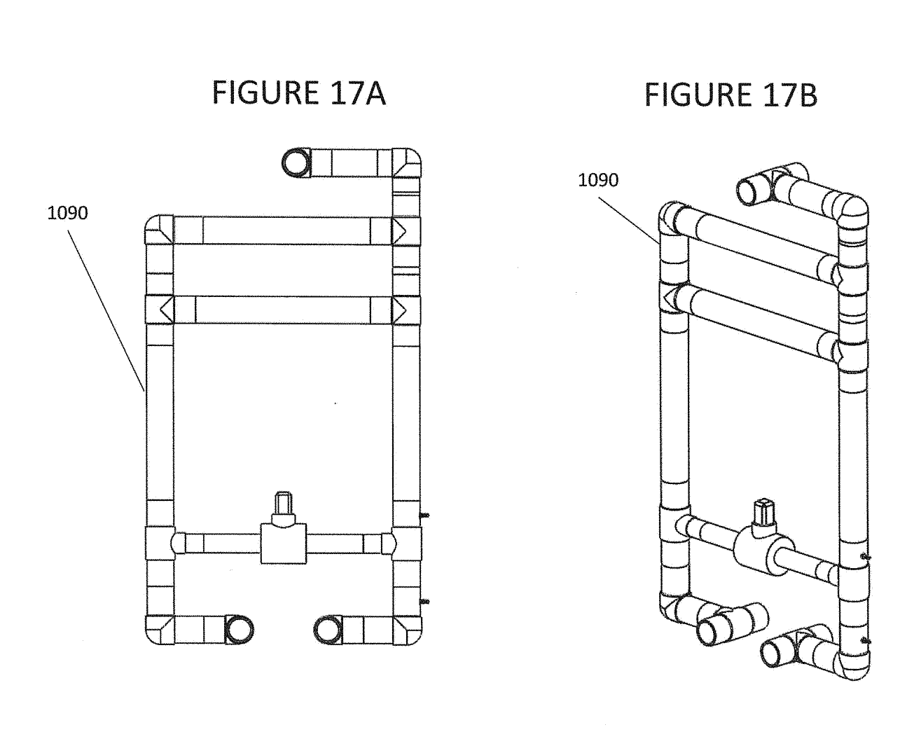

In some embodiments, as shown in FIG. 16, a photobioreactor system 1000 comprises one or more photobioreactor enclosures 1010, multiple headers connected in fluid communication with the one or more photobioreactor enclosures 1010, a conduit loop 1090 connected in fluid communication with at least one of the headers and with an external gas supply, and gas diffusers 1040 in the photobioreactor enclosures 1010. In some embodiments, a conduit loop 1090, such as shown in FIGS. 17A and 17B, is connected in fluid communication with a liquid flow in header 1022, a liquid flow out header 1024 and a gas flow in header 1026. In some embodiments, the conduit loop 1090 is positioned such that a lower portion of the conduit loop 1090 is below, and the remaining upper portion of the conduit loop 1090 is above, the height of liquid filling the photobioreactor enclosures 1010.

Orifices 1020, headers for gas and liquid flows 1030, and the conduit loop 1090 are fitted with valves, pumps, fans and other suitable fitments that enable control over flow rates and ensure that the photobioreactor enclosure 1010 can remain sealed.

It is desirable to minimize the cost of the photobioreactor system 1000 and associated supporting structures relative to the value of the product made by the liquid culture of productive organisms 1070. One of ordinary skill in the art will appreciate that suitable flexible films may be selected for use in constructing photobioreactor enclosures 1010 of the present invention for the purpose of maximizing economic efficiency. The selection of materials suitable for use in constructing the photobioreactor enclosures 1010 and supporting structures may be influenced by considerations of, for example, the total weight of liquid, gas and the liquid culture of productive organisms 1070 contained in the photobioreactor enclosure 1010 or the desired degree of translucency or light scattering for the liquid culture of productive organisms 1070.

A support structure used to maintain the vertical orientation of the photobioreactor enclosure can incorporate any suitable components and materials that minimize cost while providing adequate support and stability considering the weight of the photobioreactor enclosure and contents and considering changing environmental conditions. Such a framework can be scaled to accommodate and support multiple photobioreactor enclosures 1010 in an array. The components of the framework can comprise metal, wood, plastic, and any other materials that provide adequate strength and low cost.

During operation, the photobioreactor enclosures 1010 and the conduit loop 1090 may be filled at least partially with liquid comprising a culture of productive organisms. Liquid may be added to the photobioreactor enclosures 1010 and the conduit loop 1090 via the liquid flow in header 1022, and drained via the liquid flow out header 1024. Gas containing carbon dioxide may be added via the gas flow in header 1026, and bubbled through the gas diffuser 1040. Gas may also be added to the conduit loop 1090, in particular introduced at or near the bottom of the lower portion of the conduit loop 1090 that is filled with liquid. In some embodiments, gas is added from the same supply to the conduit loop 1090 and via the gas flow in header 1026. Gas leaves via the gas flow out header 1028. In some embodiments, liquid and gas flows are controlled using automated systems that monitor flow rates and total volumes and actuate valves, pumps and associated components in order to adjust flows.

Adding gas to the conduit loop 1090 and via the gas flow in header 1026 connected with the gas diffuser 1040 induces mixing in the partially filled photobioreactor enclosures 1010. Bubbling gas upward through liquid in the photobioreactor enclosures 1010 and in the conduit loop 1090, in conjunction with connections providing fluid communication, promotes fluid circulation throughout the photobioreactor system 1000 and mass exchange across liquid-gas interfaces.

During operation of a photobioreactor system 1000 of the present invention, in some embodiments, gas comprising carbon dioxide is bubbled through a gas diffuser 1040 into the photobioreactor enclosure 1010 to provide a feed substrate for the liquid culture of productive organisms 1070. In some embodiments, the addition of carbon dioxide is controlled to a predetermined set point for the concentration of carbon dioxide in the gas headspace 1080 above the culture.

In some embodiments, gas is vented periodically or continually from the photobioreactor enclosure 1010 to limit oxygen concentration in the gas headspace 1080 and to control gas pressure in the photobioreactor enclosure 1010 within a range that maintains proper geometry of the photobioreactor enclosure 1010 shape and structural integrity of the photobioreactor enclosure 1010. In some embodiments, the range is about 1 to about 40 inches of water. In some embodiments, the range is about 2 to about 6 inches of water.

According to the present invention, during operation of a photobioreactor system 1000 of the present invention, in photobioreactor enclosures 1010 constructed using polyethylene film, the seaming pattern shown in FIGS. 3-10 distributes mechanical stresses in a manner that maintains substantially uniform thickness of the photobioreactor enclosure 1010 and minimizes or substantially eliminates formation of touch points, folds or creases in the walls of the photobioreactor enclosure 1010 that would inhibit flows 1030 of gas and liquid.

Additionally, rounded corners in the photobioreactor enclosure 1010 with angles greater than 90 degrees, as shown in FIGS. 3-10, better facilitate flow 1030 of liquids and gases in the photobioreactor enclosure 1010 and facilitate complete flushing of the photobioreactor enclosure 1010, for example when filling or draining the photobioreactor enclosure 1010. When liquids are drained from the photobioreactor enclosure 1010, the downward slope of the seamed edges at the bottom of the photobioreactor enclosure 1010 facilitates gravity draining. When the photobioreactor enclosure 1010 is filled with liquid, the upward slope of the seamed edges at the top of the photobioreactor enclosure 1010 helps expel gas from the photobioreactor enclosure 1010 and eliminate trapped gas bubbles. The bottom and top edges of the photobioreactor 1010 enclosure may be sloped, for example, about 2% from horizontal in order to improve filling and draining of the photobioreactor enclosure 1010 with liquid and gas.

Example embodiments have been described herein for illustrative purposes only and are not limiting. Other embodiments are possible and are covered by the disclosure and the teachings contained herein. The breadth and scope of the disclosure should not be limited by any of the above-described embodiments, but should be defined only in accordance with features and claims supported by the present disclosure and their equivalents. Moreover, embodiments of the subject disclosure may include formulations, compounds, methods, systems, and devices which may further include any and all elements/features from any other disclosed formulations, compounds, methods, systems, and devices, including the manufacture and use thereof. Features from one and/or another disclosed embodiment may be interchangeable with features from other disclosed embodiments, which, in turn, correspond to yet other embodiments. One or more features/elements of disclosed embodiments may be removed and still result in patentable subject matter (and thus, resulting in yet more embodiments of the subject disclosure). Furthermore, some embodiments of the present disclosure may be distinguishable from the prior art by specifically lacking one and/or another feature, functionality, ingredient or structure, which is included in the prior art (i.e., claims directed to such embodiments may include “negative limitations” or “negative provisos”).

All references, articles, publications, patents, patent publications, and patent applications cited herein are incorporated by reference in their entireties for all purposes. Mention of any reference, article, publication, patent, patent publication, and patent application cited herein is not an acknowledgment that they constitute valid prior art or form part of the common general knowledge in any country in the world.

What is claimed is:

1. A photobioreactor for culturing productive organisms comprising an enclosure comprising a flexible film seamed in a pattern comprising:
a) an edge;
b) a channel bounded by said edge and by a plurality of linear seams disposed at a distance from the edge, wherein said linear seams are spaced apart to allow bubbles to escape the channel; and
c) a first plurality of point seams spaced apart and disposed at a distance from the plurality of linear seams, wherein the plurality of linear seams is disposed between the edge and the first plurality of point seams;
further wherein at least one portion of the flexible film is translucent.

2. The photobioreactor of claim 1, wherein the distance from the plurality of linear seams to the edge is about 2.5 inches, further wherein the horizontal spacing between adjacent point seams in the first plurality of point seams is about 2 inches, further wherein the distance from the first plurality of point seams to the plurality of linear seams is from about 1.38 inches to about 2.56 inches.

3. The photobioreactor of claim 2, wherein the pattern further comprises a second plurality of point seams spaced apart and a third plurality of point seams spaced apart, further wherein the second plurality of point seams is disposed at a distance from the first plurality of point seams, further wherein the third plurality of point seams is disposed at a distance from the second plurality of point seams, further wherein the ratio of the distance between the second plurality of point seams and the third plurality of point seams to the spacing between adjacent point seams in the second plurality of point seams is from about 0.125 to about 0.7.

4. The photobioreactor of claim 3, wherein the edge, the plurality of linear seams, the first plurality of point seams, the second plurality of point seams, and the third plurality of point seams are disposed substantially parallel.

5. The photobioreactor for culturing productive organisms of claim 1, wherein the flexible film is seamed in a pattern shown in FIG. 3.

6. A photobioreactor system comprising:
a) at least one photobioreactor enclosure of claim 1, oriented substantially vertically, wherein the seams are configured to maintain substantially uniform thickness of the at least one photobioreactor enclosure, further comprising orifices capable of permitting fluid flows into and out of the at least one photobioreactor enclosure;
b) a first header and a second header disposed below, and in fluid communication with, the at least one photobioreactor enclosure;
c) a third header and a fourth header disposed above, and in fluid communication with, the at least one photobioreactor enclosure;
d) a conduit loop in fluid communication with the first, second and third headers and with a gas supply; and
e) a gas diffuser disposed in a lower portion of the at least one photobioreactor enclosure, adapted to add gas into the at least one photobioreactor enclosure and in fluid communication with the fourth header.

7. A method of culturing productive organisms comprising the steps of:
a) inoculating a photobioreactor system of claim 6 with a culture of productive organisms;
b) exposing the culture of productive organisms to photosynthetically active radiation;
c) adding liquid to the at least one photobioreactor enclosure through the first header;
d) removing liquid from the at least one photobioreactor enclosure through the second header;
e) adding gas to the at least one photobioreactor enclosure through the fourth header and the gas diffuser, causing circulation of liquid and gas in the photobioreactor system;
f) removing gas from the at least one photobioreactor enclosure through the third header; and
g) circulating liquid and gas in the photobioreactor system.

8. The method of claim 7, wherein said circulating is achieved by adding a gas to the conduit loop.

9. The method of claim 7, wherein said circulating is achieved by means of pumping liquid through the conduit loop.
